(19) United States
(12) Patent Application Publication
(10) Pub. No.: US 2022/0176049 A1
(43) Pub. Date: Jun. 9, 2022

(54) RESPIRATORY THERAPY APPARATUS

(71) Applicant: SMITHS MEDICAL INTERNATIONAL LIMITED, Ashford (GB)

(72) Inventors: Usumah Hanif, Ashford (GB); Mohammad Qassim, Mohammad Khasawneh, Canterbury (GB); Samit K. Basu, Freemont, CA (US)

(73) Assignee: SMITHS MEDICAL INTERNATIONAL LIMITED, Ashford (GB)

(21) Appl. No.: 17/442,685

(22) PCT Filed: Mar. 20, 2020
(86) PCT No.: PCT/GB2020/000033

§ 371 Date: Sep. 24, 2021

(30) Foreign Application Priority Data
Apr. 5, 2019 (GB) 1904825.5

(51) Int. Cl. A61M 16/00; A61M 16/20

(52) U.S. Cl. CPC A61M 16/0006; A61M 16/208; A61M 2205/3375; A61M 2205/502; A61M 2205/3306

(57) ABSTRACT

An oscillatory PEP therapy device (100) has valve (10) mounted at the end of a rocker arm (12) such that the valve is opened and closed during expiration through the device, thereby causing displacement of the rocker arm up and down. The housing (2) of the device (100) is transparent so that the camera (20) on a mobile phone (200) can be positioned to view displacement of the rocker arm (12) during use. The phone (200) is programmed to extract a signal indicative of the frequency of oscillation of the rocker arm (12) and this is displayed on the screen of the phone to provide feedback to the user.

This invention relates to respiratory therapy apparatus of the kind including an oscillatory respiratory therapy device with a displaceable member displaced by breathing through the device and arranged to produce an oscillating resistance to breathing through the device.

Positive expiratory pressure (PEP) apparatus, that is, apparatus that presents a resistance to expiration through the device, are now widely used to help treat patients suffering from a range of respiratory impairments, such as chronic obstructive pulmonary disease, bronchitis, cystic fibrosis and atelectasis. More recently, such apparatus that provide an alternating resistance to flow have been found to be particularly effective. One example of such apparatus is sold under the trade mark Acapella (a registered trade mark of Smiths Medical) by Smiths Medical and is described in U.S. Pat. Nos. 6,581,598, 6,776,159, 7,059,324 and 7,699,054. Other vibratory respiratory therapy apparatus is available, such as “Quake” manufactured by Thayer, “AeroPEP” manufactured by Monaghan, “TheraPEP” manufactured by Smiths Medical and “IPV Percussionator” manufactured by Percussionaire Corp. Alternative apparatus such as “CoughAssist” manufactured by Philips is also available. Respiratory therapy apparatus can instead provide an alternating resistance to flow during inhalation.

To be effective these apparatus must be used regularly at prescribed intervals. In the case of chronic diseases, the patient needs to use the apparatus daily for the rest of his life in order to maintain a continuous relief.

Although these apparatus can be very effective, users often neglect to use the apparatus regularly at the prescribed frequency. It is very difficult to maintain a record of use of the apparatus, especially when the patient is using it at home. The clinician often does not know whether deterioration in a patient's condition is because he has failed to use the apparatus as prescribed or whether other factors are the cause. WO2014/202923 describes apparatus including an acoustic sensor such as a microphone responsive to the sound produced by the therapy device. Other apparatus is described, for example, in WO2014/202924, WO2015/036723, WO2015/104522, WO2015/114285, WO2015/198001, WO2016/075426, WO2016/092247, WO2017/178776, WO2017/194906, WO2017/187116, GB2560105 and WO2019/243758.

It is an object of the present invention to provide alternative respiratory therapy apparatus.

According to one aspect of the present invention there is provided a respiratory therapy apparatus of the above-specified kind, characterised in that the apparatus includes an optical sensor responsive to displacement of the displaceable member.

The optical sensor is preferably arranged to provide a signal indicative of one or more of the following: when the device is used, the duration of use, and the frequency of oscillation. The optical sensor is preferably provided externally of the device, the device having a transparent housing or a transparent window through which the sensor can view the displaceable member in the device. The optical sensor may contained in a unit including a display on which is represented the frequency of oscillation detected by the sensor. The optical sensor may be provided by an electronic camera. The electronic camera may be provided in a phone, the phone being programmed to respond to displacement of the displaceable member in the device during use. The displaceable member may include a valve element on a rocker arm that opens and closes an opening during exhalation through the device. A part at least of the displaceable member may be marked with a contrasting colour or a reflective surface to improve sensitivity of the optical sensor to displacement of the displaceable member. The apparatus may also include a microphone arranged to sense the sound made by the therapy device, the apparatus combining signals from the optical sensor and microphone to improve the signal to noise ratio. The optical sensor may be arranged to provide an indication of the range of the therapy device from the sensor, the gain of the apparatus being adjusted accordingly.

According to another aspect of the present invention there is provided an optical sensor for use with apparatus according to the above one aspect of the present invention.

According to a further aspect of the present invention there is provided a phone with a camera and programmed for use with apparatus according to the above one aspect of the present invention.

According to a fourth aspect of the present invention there is provided a respiratory therapy device in the form of an oscillatory PEP device for use with apparatus according to the above one aspect of the present invention.

Apparatus including an oscillatory PEP device will now be described, by way of example, with reference to the accompanying drawings, in which:

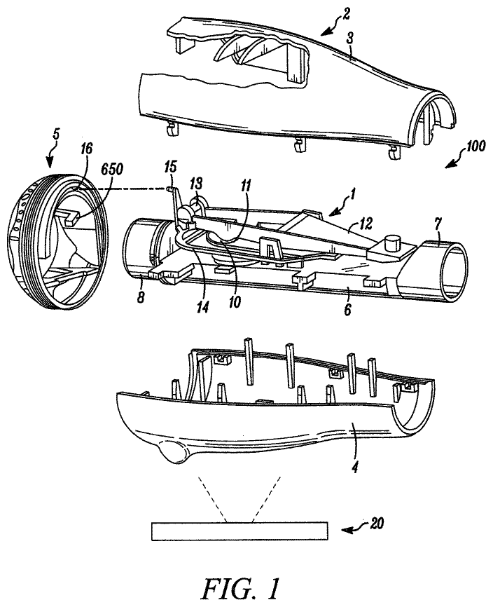

FIG. 1 is an exploded view of the apparatus;

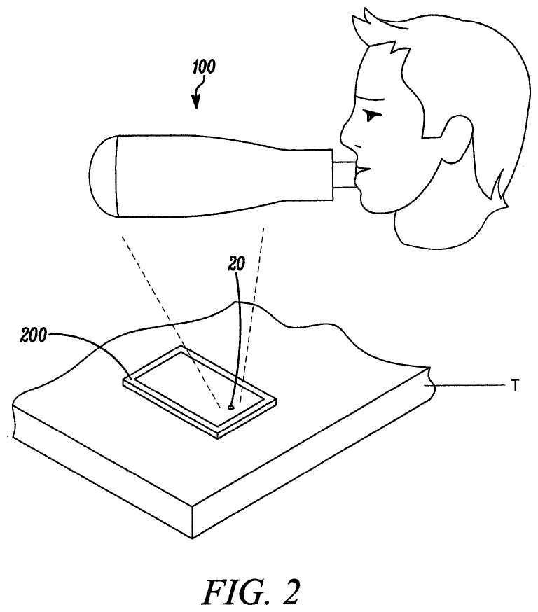

FIG. 2 illustrates the apparatus in use; and

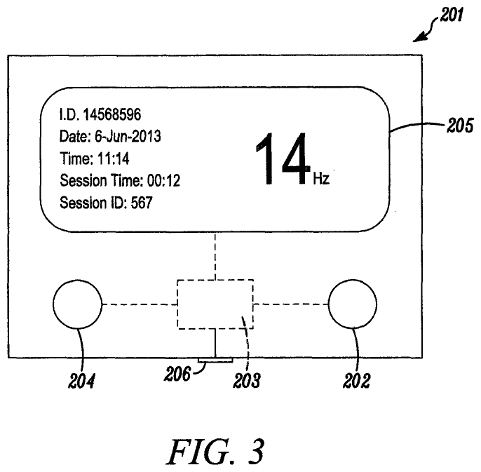

FIG. 3 is a front elevation view of one form of a dedicated sensor.

With reference first to FIG. 1, the device 100 comprises a rocker assembly 1 contained within an outer, optically-transparent housing 2 provided by an upper part 3 and a lower part 4 of substantially semi-cylindrical shape. The device is completed by an adjustable dial 5 of circular section. The rocker assembly 1 includes an air flow tube 6 with a breathing inlet 7 at one end and an inspiratory inlet 8 at the opposite end including a one-way valve (not shown) that allows air to flow into the air flow tube 6 but prevents air flowing out through the inspiratory inlet. The air flow tube 6 has an outlet opening 10 with a non-linear profile that is opened and closed by a conical valve element 11 mounted on a rocker arm 12 pivoted midway along its length about a transverse axis. The air flow tube 6 and housing 2 provide a structure with which the rocker arm 12 is mounted. At its far end, remote from the breathing inlet 7, the rocker arm 12 carries an iron pin 13 that interacts with the magnetic field produced by a permanent magnet (not visible) mounted on an adjustable support frame 14. The magnet arrangement is such that, when the patient is not breathing through the device, the far end of the rocker arm 12 is held down such that its valve element 11 is also held down in sealing engagement with the outlet opening 10. A cam follower projection 15 at one end of the support frame 14 locates in a cam slot 16 in the dial 5 such that, by rotating the dial, the support frame 14, with its magnet, can be moved up or down to alter the strength of the magnetic field interacting with the iron pin 13. The dial 5 enables the frequency of operation and the resistance to flow of air through the device to be adjusted for maximum therapeutic benefit to the user.

When the patient inhales through the breathing inlet 7 air is drawn through the inspiratory inlet 8 and along the air flow tube 6 to the breathing inlet. When the patient exhales, the one-way valve in the inspiratory inlet 8 closes, preventing any air flowing out along this path. Instead, the expiratory pressure is applied to the underside of the valve element 11 on the rocker arm 12 causing it to be lifted up out of the opening 10 against the magnetic attraction, thereby allowing air to flow out to atmosphere. The opening 10 has a non-linear profile, which causes the effective discharge area to increase as the far end of the rocker arm 12 lifts, thereby allowing the arm to fall back down and close the opening. As long as the user keeps applying sufficient expiratory pressure, the rocker arm 12 will rise and fall repeatedly as the opening 10 is opened and closed, causing a vibratory, alternating or oscillating resistance to expiratory breath flow through the device. Further information about the construction and operation of the device can be found in U.S. Pat. No. 6,581,598, but is not essential for an understanding of the present invention.

As so far described, the apparatus is conventional.

The apparatus of the present invention includes the device 100 described above and an optical sensor 20 responsive to movement of the rocker arm 12 caused breathing through the device.

FIG. 2 shows a device 100 and an optical sensor provided by an electronic camera 20 in a conventional mobile phone 200. As shown, the phone 200 might conveniently be placed on the top of a table T at which the user is sitting so that it lies beneath the therapy device 100 when this is held up to the mouth during use.

FIG. 3 shows an alternative optical sensor provided by a stand-alone camera unit 201 that is separate from the device 100 but, in use, is placed close to it. The camera unit 201 includes a conventional electronic optical video sensor 202 connected to a processing and memory unit 203, which is also connected to an on/off button 204 and a display screen 205. The unit 201 also has a data interface, such as the USB port 206 shown, or a wireless interface, such as a radio frequency Bluetooth or an infra-red interface. The unit 201 may also include input means by which information can be entered to the sensor unit, or this could be carried out via the data interface 206 from a remote computer or the like.

In use, the mobile phone 200 or stand-alone camera unit 201 is placed close to the device 100, so that displacement of the rocker arm 12 is within the visible range of the camera, but is not in direct contact with the device. Typically, the phone 200 or other unit 201 would be placed within about 25 cm of the therapy device 100. The stand-alone unit 201 is turned on using the button 204, thereby causing the display 205 to show a representation of the user's identification, such as in the form of a unique number, the date and present time. When the user starts the therapy session the rocker arm 12 moves up and down as the user exhales through the device 100. The output of the camera unit 201 is appropriately processed by the processing unit 203 to extract a signal indicative of the frequency of oscillation of the rocker arm 12. The processing unit 203 can measure various parameters, such as the duration of each exhalation, the number of exhalations in each session, and the oscillation frequency during exhalation. As illustrated in FIG. 3, the screen 205 provides the user with a representation of the session time and the frequency of oscillation, which has been found to be particularly important to users in achieving the best therapeutic effect. The sensor unit 201 or phone 200 preferably also provides the user with feedback as to whether he has achieved his target oscillation frequency. This may be done in various different ways, such as by displaying a legend on the display: “Flow OK”, “Flow Too High” or “Flow Too Low”. Alternatively, the screen 205, or a part of the screen, could change colour to indicate whether flow was too high or low, or a sound signal could be produced.

As mentioned above, the setting of the dial 5 on the therapy device 100 affects the frequency and resistance to flow through the device. This is set by the user to achieve the maximum beneficial effect. The sensor unit 200, 201 could be arranged to compute a measure of the flow rate and pressure generated from the measured frequency and from knowledge of the setting of the dial 5, as entered into the sensor unit by the user or clinician.

The camera sensor unit 200, 201 can be tuned to be sensitive to oscillations in a relatively small range of frequencies. By filtering out other frequencies it is possible to use high gain amplification for maximum sensitivity.

The phone 200 and sensor unit 201 described above are both separate from the therapy device 100. However, a sensor unit could be mounted with the therapy device, such as by means of a clip or strap that supports the sensor on the device. The sensor unit need not include a camera but could include a simple light sensor responsive to light reflected by the rocker arm or some other moving component. Alternatively, the optical sensor unit could include a simple optical sensor and a light source positioned on opposite sides of the rocker arm 12 or other movable member such that a beam of light from the source is alternately interrupted or passed by the rocker arm to the sensor.

The optical sensor need not be responsive to radiation in the visible part of the spectrum but could be responsive to other optical radiation, such as infra-red or ultra-violet radiation. Where the optical sensor needs to respond to movement of the displaceable member within the housing of the therapy device it will be appreciated that the housing, or a window, would need to be transparent to the radiation being sensed, that is, to visible, infra-red or ultra-violet radiation.

Instead of being incorporated into a mobile phone, the sensor unit could be provided as a program application in a general purpose computer, using the camera built into the computer, or a separate plug-in camera. The program application could be arranged to stop automatically after the elapse of a predetermined time without sensing any movement having the appropriate characteristics.

In the arrangement described above the camera or other optical sensor is positioned to sense the up and down rocking movement of the rocker arm 12 through the transparent housing 2. It is not essential that the housing be entirely transparent if there is a transparent window through which the rocker arm or other displaceable member is visible. In order to improve sensitivity of the optical sensor it might be desirable to mark a part or all of the rocker arm with a contrasting colour or a reflective surface. Alternatively, the displaceable member in the therapy device could be mechanically linked to a flag on the outside the casing of the device so that the flag moves with the displaceable member and can be viewed by the optical sensor.

It will be appreciated that there are many different ways in which information obtained by the sensor unit can be represented so that it is provided to the user and clinician in the most useful manner.

Apparatus of the present invention can be used with any conventional oscillatory respiratory therapy device having a displaceable member the movement of which can be viewed optically either directly or indirectly. The therapy device may be combined with other treatments such as nebulisation or the administration of aerosol medication.

The optical sensor could be combined with an arrangement including a microphone that senses the sound made by the therapy device, such as of the kind described in WO2014/202923. By combining the optical and acoustic signals the signal to noise ratio of the apparatus can be improved. The optical sensor could be used to provide an indication of the range of the therapy device from the sensor, the apparatus being arranged to adjust its gain appropriately.

Where the optical sensor is separate from the therapy device it enables the device to be cleaned periodically without the risk of damaging any electrical components in the sensor. It also enables the device to be disposed of at the end of its life without the need to dispose of the optical sensor, which can be reused with a replacement therapy device.

The present invention enables apparatus to be provided that gives useful data about use of an existing, conventional therapy device with little or no modification of the device itself. In this way, the user can be made more aware of how well he is complying with the prescribed therapy programme and can modify his use of the device accordingly to achieve maximum benefit. The clinician is also able to check patient compliance so that he can identify whether any deterioration in a patient's condition is due to lack of compliance or if alternative treatment is needed. The therapy device need not be an expiratory therapy device but could, for example be an inspiratory therapy device.

1-13. (canceled)

14. Respiratory therapy apparatus including an oscillatory respiratory therapy device with a displaceable member displaced by breathing through the device and arranged to produce an oscillating resistance to breathing through the device, characterised in that the apparatus includes an optical sensor responsive to displacement of the displaceable member.

15. The respiratory therapy apparatus according to claim 14, characterised in that the optical sensor is arranged to provide a signal indicative of one or more of the following: when the device is used, the duration of use, and the frequency of oscillation.

16. The respiratory therapy apparatus according to claim 14, characterised in that the optical sensor is provided externally of the device, and that the device has a transparent housing or a transparent window through which the sensor can view the displaceable member in the device.

17. The respiratory therapy apparatus according to claim 14, characterised in that the optical sensor is contained in a unit including a display on which is represented the frequency of oscillation detected by the sensor.

18. The respiratory therapy apparatus according to claim 14, characterised in that the optical sensor is provided by an electronic camera.

19. The respiratory therapy apparatus according to claim 18, characterised in that the electronic camera is provided in a phone, and that the phone is programmed to respond to displacement of the displaceable member in the device during use.

20. The respiratory therapy apparatus according to claim 14, characterised in that the displaceable member includes a valve element on a rocker arm that opens and closes an opening during exhalation through the device.

21. The respiratory therapy apparatus according to claim 14, characterised in that a part at least of the displaceable member is marked with a contrasting colour or a reflective surface to improve sensitivity of the optical sensor to displacement of the displaceable member.

22. The respiratory therapy apparatus according to claim 14, characterised in that the apparatus also includes a microphone arranged to sense the sound made by the therapy device, and that the apparatus combines signals from the optical sensor and the microphone to improve the signal to noise ratio.

23. The respiratory therapy apparatus according to claim 22, characterised in that the optical sensor is arranged to provide an indication of the range of the therapy device from the sensor, and that the apparatus is arranged to adjust its gain accordingly.

24. The respiratory therapy apparatus according to claim 14, characterised in that a phone with a camera is adapted to be programmed for use with the apparatus.

25. The respiratory therapy apparatus according to claim 14, characterised in that the respiratory therapy device is in the form of an oscillatory PEP device.

26. An optical sensor for use with an apparatus having an oscillatory respiratory therapy device with a displaceable member displaced by breathing through the device and arranged to produce an oscillating resistance to breathing through the device, characterised in that the optical sensor is responsive to displacement of the displaceable member in the oscillatory respiratory therapy device.
